(19) United States
(12) Patent Application Publication
(10) Pub. No.: US 2024/0207321 A1
(43) Pub. Date: Jun. 27, 2024

(54) METHODS FOR REPROGRAMMING TARGET CELLS

(71) Applicant: Duke University, Durham, NC (US)

(72) Inventors: Anthony Filiano, Durham, NC (US); Hyunjung Min, Durham, NC (US); Joanne Kurtzberg, Durham, NC (US); Li Xu, Durham, NC (US)

(73) Assignee: Duke University, Durham, NC (US)

(21) Appl. No.: 18/024,091

(22) PCT Filed: Sep. 8, 2021
(86) PCT No.: PCT/US21/49428

§ 371 Date: Mar. 1, 2023

Related U.S. Application Data
(60) Provisional application No. 63/076,021, filed on Sep. 9, 2020.

(51) Int. Cl. A61K 35/28

(52) U.S. Cl. CPC A61K 35/28

(57) ABSTRACT

The present disclosure provides, in part, a method of reprogramming a target cell using mesenchymal stromal stem cells (MSCs). Also provided are various uses of such method for medical treatments. Further provided are a pharmaceutical composition comprising processing bodies (p-bodies) and uses thereof.

### CROSS REFERENCE TO RELATED APPLICATIONS

This application is a national phase application of PCT Application No. PCT/US2021/049428 filed Sep. 8, 2021, which claims benefit of priority to U.S. Provisional Patent Application No. 63/076,021, filed Sep. 9, 2020, the contents of each of which are hereby incorporated by reference in their entirety.

### INCORPORATION BY REFERENCE OF SEQUENCE LISTING PROVIDED ELECTRONICALLY

This application contains a Sequence Listing submitted as an electronic TXT file named “21-2027-WO-US”, having a size in bytes of 1 KB and created on Jul. 16, 2023. The information contained in this electronic file is incorporated herein by reference in its entirety.

### FIELD

The present disclosure generally relates to medical treatments. In particular, the present disclosure relates to methods of reprogramming target cell(s) and various medical uses thereof.

### BACKGROUND

Mesenchymal stromal cells (MSCs) are widely used in clinicals trial due to their ability to modulate inflammation. The success of MSCs has been variable over 25 years likely due to an incomplete understanding of their mechanism. After MSCs are administered, they travel to the lungs and other tissues where they are rapidly cleared. Despite being cleared, MSCs suppress the inflammatory response long-term. There is a need to understand how MSCs modulate inflammation and hence a need for methods to enhance the immunomodulator capabilities of MSCs.

### SUMMARY

The present disclosure is based, in part, on a mechanism newly discovered as to how the umbilical cord derived mesenchymal stromal stem cells (MSCs) reprogram myeloid cells to suppress a T cell response. Myeloid cells such as monocytes or macrophages engulf cytoplasmic components of MSCs and undergo transcriptional reprogramming. The interaction between the myeloid cells and the MSCs depends on lipoprotein receptor-related proteins (LRPs) on the myeloid cells and is mediated by processing bodies (“p-bodies”) within MSCs. This discovery uncovers potential therapeutic targets to enhance the immunomodulator capabilities of MSCs.

Accordingly, one aspect of the present disclosure provides a method of reprogramming a target cell. Such method comprises contacting the target cell with one or more mesenchymal stromal stem cells (MSCs).

In some embodiments, the MSCs are derived from umbilical cord tissue, bone marrow, adipose tissue, and/or induced pluripotent stem cells (iPSCs).

In some embodiments, the target cell is a myeloid cell or a population of myeloid cells. By way of non-limiting example, the myeloid cell is a monocyte, a macrophage, or a dendritic cell.

In some embodiments, the myeloid cell engulfs the MSCs via cell-to-cell interaction between the myeloid cell and the MSCs.

In some embodiments, the method disclosed above and herein further comprises manipulating the MSCs to target the delivery of the cytoplasmic components of the MSCs to the myeloid cell.

In some embodiments, the cytoplasmic components are processing bodies (p-bodies) within the MSCs.

In some embodiments, the cell-to-cell interaction is mediated through lipoprotein receptor-related proteins (LRPs) on the surface of the myeloid cell.

Another aspect of the present disclosure provides a method of suppressing a T cell response in a subject in need thereof. Such method comprises reprogramming a target cell in the subject according to any of the methods disclosed above and herein.

In some embodiments, activation of helper T cells is suppressed in the subject.

Another aspect of the present disclosure provides a method of reducing or inhibiting an immune response in a subject in need thereof. Such method comprises reprogramming a target cell in the subject according to any of the methods disclosed above and herein.

Another aspect of the present disclosure provides a method of reducing or inhibiting an inflammatory response in a subject in need thereof. Such method comprises reprogramming a target cell in the subject according to any of the methods disclosed above and herein. By way of non-limiting example, the subject suffers from a lung inflammation, neuroinflammation, rheumatoid arthritis, and/or a primary immunodeficiency.

Another aspect of the present disclosure provides a method of reducing an immune response to a gene therapy regime in a subject in need thereof. Such method comprises reprogramming a target cell in the subject according to any of the methods disclosed above and herein.

Still another aspect of the present disclosure provides a pharmaceutical composition comprising processing bodies (p-bodies).

In some embodiments, the p-bodies are present within or isolated from mesenchymal stromal cells (MSCs). In some embodiments, the MSCs are derived from umbilical cord tissue, bone marrow; adipose tissue, and/or induced pluripotent stem cells (iPSCs).

In some embodiments, the pharmaceutical composition further comprises a therapeutically acceptable carrier.

Still another aspect of the present disclosure provides a method of reprogramming a target cell by contacting the target cell with the pharmaceutical composition disclosed above and herein.

In some embodiments, the target cell is a myeloid cell or a population of myeloid cells. By way of non-limiting example, the myeloid cell is a monocyte, a macrophage, or a dendritic cell.

Still another aspect of the present disclosure provides a method of suppressing a T cell response in a subject in need thereof. Such method comprises administering to the subject a therapeutically effective amount of the pharmaceutical composition disclosed above and herein.

In some embodiments, activation of helper T cells is suppressed in the subject.

Still another aspect of the present disclosure provides a method of reducing or inhibiting an immune response in a subject in need thereof. Such method comprises administering to the subject a therapeutically effective amount of the pharmaceutical composition disclosed above and herein.

Still another aspect of the present disclosure provides a method of reducing or inhibiting an inflammatory response in a subject in need thereof. Such method comprises administering to the subject a therapeutically effective amount of the pharmaceutical composition disclosed above and herein. By way of non-limiting example, the subject suffers from a lung inflammation, neuroinflammation, rheumatoid arthritis, and/or a primary immunodeficiency.

Still another aspect of the present disclosure provides a method of reducing an immune response to a gene therapy regime in a subject in need thereof. Such method comprises reprogramming a target cell in the subject according to any of the methods disclosed above and herein.

Further aspect of the present disclosure provides a kit comprising the pharmaceutical composition disclosed above and herein and an instruction manual.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and other features of the invention are explained in the following description, taken in connection with the accompanying drawings, wherein:

FIG. 1A shows cord tissue isolated MSCs express canonical MSC markers. In vitro cultured MSCs obtained from cord tissue were collected and stained using anti-CD73, CD90, CD105, CD166, CD44, CD45 and CD31 and then analyzed using FACSCanto™. FIG. 1B shows hCT-MSCs were differentiated to adipocyte, osteoblast or chondrocyte and each type of cells were stained with FABP4 (adipocyte), osteocalcin (osteocyte), or alcian blue (chondrocyte).

FIGS. 2A-2F are graphs showing hCT-MSCs program monocytes and macrophages to inhibit the activation of T cells. FIG. 2A shows hCT-MSCs inhibit the proliferation of T cells from human PBMCs. T cell proliferation was measured in counts/minute by the incorporation of 3H-thymidine (1-way ANOVA; post-hoc *** p<0.001; N=3). FIG. 2B shows TH cell proliferation assay. T cells were stained with CFSE and proliferation of CD4+TH cells was measured by CFSE dilution after stimulated with anti-CD3/28. hCT-MSCs block TH cell proliferation in a population of splenocytes (open bars) but not isolated T cells (black bars; 2-way ANOVA; post-hoc * p<0.05). FIG. 2C shows whole human PBMC or T cells from human PBMC were stained with CFSE and cocultured with hCT-MSC with anti-CD3/28. 3 days after, cells were stained using anti-CD4 and TH cell proliferation was measured by CFSE dilution (black bars; 2-way ANOVA; post-hoc * p<0.05). FIG. 2D shows experimental schematic for programing monocytes and macrophages. FIG. 2E shows hCT-MSCs physically interact with mouse monocytes and macrophages, then monocytes and macrophages can suppress T cells when transferred to a new well (red bar). Conversely, if monocytes and macrophages were cultured in MSC expansion media without being exposed to hCT-MSCs (CTL grey bar) or are blocked from directly interacting with hCT-MSCs, via a transwell membrane (blue bar), they no longer suppress T cells (1-way ANOVA; post-hoc *** p<0.001). FIG. 2F shows T cell depleted human PBMCs were preconditioned by hCT-MSC coculture in same well (red bar) or in the transwell to block direct cell-to-cell contact (blue bar). Control (CTL; grey bar) T cell depleted PBMCs were incubated in MSC expansion media without hCT-MSCs. Preconditioned cells were transferred to CFSE labelled isolated human T cell with anti-CD3/28 and proliferation of TH cell were measured by CFSE dilution (1-way ANOVA; post-hoc *** p<0.001; N=3).

FIGS. 3A-3C show mouse splenocyte were cocultured with hCT-MSC and stimulated with anti-CD3/28 for 24 h. And then, cells were collected, stained with anti-CD4, CD25, CD69 and the signals were measured using BD FACSLyric™ (1-way ANOVA; post-hoc * p<0.05, ** p<0.01; N=3). FIG. 3D shows CFSE labelled mouse T cells were cocultured with HUVEC cells and stimulated with anti-CD3/28. T cell proliferation was measured by the dilution of CFSE. FIG. 3E shows after pre-incubating with hCT-MSCs, T cell-depleted splenocytes were either directly added to a T cell proliferation assay (0 h) or cultured for 24 or 48 h prior to adding to a T cell proliferation assay. TH proliferation was measured by CFSE dilution (bottom grey bar; 1-way ANOVA; post-hoc *** p<0.001; ** p<0.01; N=3) in accordance with one embodiment of the present disclosure.

FIG. 4A shows T cell-depleted splenocytes were cocultured with MSCs for 3 days and stained with anti-CD73 and CD90. FIG. 4B shows as a control, adherent cells (MSCs) were collected and stained using the same antibodies. Cells were analyzed using the BD FACSLyric™.

FIGS. 5A-5G are graphs and images showing monocytes and macrophages that engulf hCT-MSCs are programmed to inhibit T cells in accordance with one embodiment of the present disclosure. FIG. 5A shows hCT-MSCs were labeled with Qtracker (cytoplasmic stain) or Edu (nuclei stain) then incubated with macrophages. Monocytes and macrophages take up Qtracker but not Edu. Qtracker and EdU signal were analyzed by flow cytometry (representative histograms) and the percent of monocytes and macrophages that are Qtracker positive were quantified (t-test **p<0.01; N=3). FIG. 5B shows representative confocal z-stacks and 3D reconstruction of hCT-MSCs labelled with Qtracker and cocultured with GFP+ monocytes and macrophages. FIG. 5C shows T cell-depleted splenocytes were cocultured with Qtracker labelled hCT-MSCs and 2 days afterwards, cells were stained with anti-B220, CD11b, CD11c, F4/80, Ly6C, Ly6G to analyze the cell type which engulfed hCT-MSCs (Macrophage: CD11b+F4/80+Ly6G−; Monocyte LyChi: CD11b+F4/80−Ly6G− Ly6hi; Monocyte Ly6lo: CD11b+F4/80−Ly6G− Ly6lo; B cell: CD11b−B220+ Dendritic Cell: CD11b−CD11c; Neutrophil: CD11b+Ly6G+; (t-test * p<0.05, ** p<0.01; N=3)). FIG. 5D shows representative histograms of CD11b+Ly6G− cells after incubating with Qtracker/EdU labelled cells (live or apoptotic hCT-MSCs and thymocytes). FIG. 5E shows T cell proliferation assay. CD11b+ cells were isolated from collected splenocytes and cocultured with CFSE-labelled T cell with anti-CD3/28 stimulation for 3 days. CFSE signal of T cells was measured using BD FACSLyric™ (1-way ANOVA; post-hoc * p<0.05, ** p<0.01; N=3). FIG. 5F shows T cell-depleted splenocytes were cocultured with Qtracker labelled MSC for 2 days and stained using anti-CD11b and Ly6G. Qtracker positive/negative CD11b+, Ly6G− cells were sorted using MoFlo Astrios Cell Sorter. FIG. 5G shows Monocytes and macrophages were FACS sorted on their ability to engulf hCT-MSCs (i.e., Qtracker positive and negative), then tested for their ability to inhibit T cells. T cell proliferation was measured by CFSE dilution (1-way ANOVA; post-hoc *** p<0.001; N=3).

FIG. 6 shows T cell-depleted mouse splenocytes were cocultured with Qtracker labelled hCT-MSCs and 2 days afterwards, cells were stained with anti-B220, CD11b, CD11c, F4/80, Ly6C, Ly6G and analysed by BD FACSLyric™.

FIG. 7 shows hCT-MSCs were treated with 10 μg/mL cycloheximide with 20 ng/ml TNF-α for 24 hours to induce apoptosis. Apoptotic cells were collected and cocultured with T cell-depleted splenocytes for 2 days, and after splenocytes were collected, adherent cell populations were collected and stained with anti-CD90, CD73 and 7AAD. Cells were analyzed using the BD FACSLyric™.

FIGS. 8A-8F are graphs showing the transcriptional changes of monocytes and macrophages after engulfing cytoplasmic components of hCT-MSCs. (A-E) After 2 day-coculture with Qtracker labelled hCT-MSC. mouse splenocytes were collected and Qtracker positive/negative CD11b+, Ly6G− cells were sorted using MoFlo Astrios Cell Sorter. RNAs were collected from each cells for RNA sequencing. FIG. 8A shows PCA analysis of Q+ and Q− monocytes and macrophages. FIG. 8B shows gene sets enriched in Qtracker positive (Q+) and Qtracker negative (Q−) monocytes and macrophages. FIG. 8C shows Qtracker positive monocytes and macrophages up-regulate genes associated with phagocytosis and down-regulated genes associated with T cell activation or proliferation. FIG. 8D shows Qtracker positive monocytes and macrophages down-regulated associated with antigen presentation and co-stimulation. FIG. 8E shows genes related with diseases differentially expressed in Qtracker positive monocytes and macrophages. FIG. 8F shows expression of mRNAs related with antigen presentation were decreased in Q+ monocytes and macrophages.

FIGS. 9A-9C are graphs showing monocytes and macrophages interact with MSCs through LRP in accordance with one embodiment of the present disclosure. FIG. 9A shows genes that express receptors mediating cell-to-cell interactions were curated and plotted for differential expression upregulated in monocytes and macrophages that engulf cytoplasmic components of MSCs. The top 7 upregulated and lowest adjusted p-value is marked in red. Inhibitors for 6 of 7 receptors were tested in the Qtracker MSC engulfment assay, including RAP, a pan inhibitor of LRPs. FIG. 9B shows representative histogram of Qtracker positive macrophages (N=3). FIG. 9C shows quantification of the percent of monocytes and macrophages that are Qtracker positive (1-way ANOVA; post-hoc *** p<0.001; N=3).

FIG. 10 shows T cell depleted mouse splenocyte were incubated with blocker for MertK, TREM2 or ITGA9 for 1 hour and then cocultured with QTracker labelled hCT-MSC. QTracker signal in macrophage were analysed using BD FACSLyric™. Concentrations used were 10× and 100×.

FIGS. 11A-11H are images and graphs showing P-bodies are needed to program macrophages to inhibit TH cell activation. FIG. 11A shows representative confocal image of hCT-MSCs stained with DCP1A and DDX6 antibodies. FIG. 11B shows representative image of hCT-MSC transiently transfected with DDX6-RFP obtained by confocal. FIG. 11C shows 3D reconstruction of monocytes or macrophages which engulfed DDX6-RFP from hCT-MSC. FIG. 11D shows Western blot analysis was performed to detect DDX6 protein expression in control hCT-MSCs and DDX6 KO hCT-MSCs (t-test *** p<0.001; N=3). FIG. 11E shows representative confocal image and Dcp1a puncta counts of Control and DDX6-KO hCT-MSCs transiently transfected with DCP1a-GFP (N=5). FIG. 11F shows CFSE labelled mouse splenocytes were cocultured with control or DDX6 KO MSCs and stimulated with anti-CD3/28 (1-way ANOVA; post hoc * p<0.05; N=3). FIG. 11G shows T cell-depleted mouse splenocytes were preconditioned by control or DDX6 KO MSC for 3 days. Then, preconditioned cells were transferred to CFSE-labelled T cell stimulated with anti-CD3/28 (1-way ANOVA; post-hoc ** p<0.01; N=3). FIG. 11H shows whole human PBMC were stained with CFSE and cocultured with control or DDX6 KO hCT-MSCs then stimulated with anti-CD3/28. 3 days after, cells were stained using anti-CD4 and T cell proliferation was measured by CFSE dilution (1-way ANOVA; post-hoc * p<0.05; N=3).

FIG. 12A shows control hCT-MSCs and DDX6 KO hCT-MSCs were labelled with Qtracker and each cell was cocultured with T cell-depleted splenocytes for 2 days. Cells were stained with anti-CD11b and Ly6G. Qtracker signal in CD11b+Ly6G− cells was measured using the BD FACSLyric™. FIG. 12B shows viability of Control hCT-MSCs and DDX6 KO hCT-MSCs after co-culturing with splenocytes for 3 days. Cells were stained with anti-CD73, CD90 and 7AAD in accordance with one embodiment of the present disclosure.

FIG. 13A shows cells were isolated from lungs of mice and stained with anti-CD3, CD11b, CD11c, Siglec-F. I-A/I-E, and 7AAD. Cells were then analyzed using BD FACSLyric™. FIG. 13B shows representative histograms of QTracker positive monocytes and macrophages isolated from the lung 24 after injecting hCT-MSCs (IV).

FIGS. 14A-14C are graphs showing P-bodies are needed to suppress monocytes and macrophages during lung inflammation. Control or DDX6 KO hCT-MSCs were injected IV 2 hours prior to mice receiving intranasal LPS to induce lung inflammation. Cells were isolated from lung and stained with anti-CD3, CD11b, CD11c, I-A/I-E and 7AAD then analyzed by flow cytometry. FIG. 14A shows CD11c+/CD11c−/Siglec-F+ Alveolar macrophage populations from lungs (1-way ANOVA; post hoc * p<0.05; N=3-4). FIG. 14B shows CD11b+monocytes and macrophage populations from lungs. (1-way ANOVA; post hoc * p<0.05; ** p<0.01; N=3-4). FIG. 14C shows MHC class II expression on lung CD11b+/CD11c− monocytes and macrophages were measured by FACS flow (1-way ANOVA; post hoc * p<0.05; *** p<0.001; N=3-4).

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of non-limiting example, “an element” means at least one element and can include more than one element.

“About” is used herein to provide flexibility to a numerical range endpoint by providing that a given value may be “slightly above” or “slightly below” the endpoint without affecting the desired result.

As used herein, the term “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “administering” an agent, such as a therapeutic entity (e.g., an MSC or the like) to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery and administration by the intranasal or respiratory tract route.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

As used herein, the term “reprogramming”, “reprogram” or “transcriptional reprogramming” refers to altering the RNA transcripts expressed by the target cell(s).

Unless otherwise defined, all technical terms used herein have the same meaning as commonly understood by one of ordinary skill in the art to which this disclosure belongs.

The present disclosure demonstrates that human cord tissue-derived MSCs (hCT-MSCs) directly interacted and reprogrammed monocytes and macrophages. After engaging hCT-MSCs, monocytes and macrophages engulfed cytoplasmic components of live hCT-MSCs then down-regulated gene programs for antigen presentation and co-stimulation and functionally suppressed the activation of helper T cells. It was determined that low density lipoprotein receptor-related proteins (LRP) on the surface of monocytes and macrophages mediated the engulfment of hCT-MSCs. Since a large amount of cellular information can be packaged in cytoplasmic RNA processing bodies (“p-bodies”; intracellular organelles used to maintain RNAs), p-body deficient hCT-MSCs were generated and it was confirmed that they failed to reprogram monocytes and macrophages in vitro and in vivo. Overall, a novel mechanism was uncovered where hCT-MSCs indirectly suppressed a T cell response by directly interacting and reprogramming monocytes and macrophages via p-bodies. This discovery implicates a novel mechanism how MSCs can reprogram the inflammatory response and have long-term effects to suppress inflammation.

Accordingly, one aspect of the present disclosure provides a method of reprogramming a target cell. Such method comprises contacting the target cell with one or more mesenchymal stromal stem cells (MSCs). In some embodiments, this method includes direct contact of target cells with MSCs. The MSCs may be derived from human tissues and/or cells, including, but not limited to, umbilical cord tissue (hCT-MSCs), bone marrow, adipose tissue, and induced pluripotent stem cells (iPSCs).

In some embodiments, the target cell is a myeloid cell or a population of myeloid cells. The myeloid cell includes, but is not limited to, a monocyte, a macrophage, and a dendritic cell.

The myeloid cell engulfs the MSCs by cell-to-cell interaction between the myeloid cell and the MSCs. The cell-to-cell interaction is mediated through lipoprotein receptor-related proteins (LRPs) on the surface of the myeloid cell such as monocytes and macrophages. Upon contact, the monocytes and/or macrophages engulf cytoplasmic components of the MSCs and undergo transcriptional reprogramming. Thus, in some embodiments, the cell-to-cell interaction is direct.

That is, in the method disclosed above and herein, the MSCs may be applied to the target cells directly or the cell-to-cell interaction between the target cells (e.g., monocytes, macrophages) and the MSCs is direct.

In some embodiments, the method disclosed above and herein may further comprise manipulating the MSCs to target the delivery of the cytoplasmic components of the MSCs to the myeloid cell. The cytoplasmic components engulfed by the myeloid cell are processing bodies (p-bodies) within the MSCs.

The discovery of the present disclosure of how hCT-MSCs reprogrammed myeloid cells led to potential therapeutic targets to enhance the immunomodulator capabilities of MSCs. These targets include, but are not limited to, the manipulation of MSCs to increase the amount and manipulate the components of p-bodies; the manipulation of MSCs to target the delivery of p-bodies; and the isolation and packaging of p-bodies for a stand-alone product, among others.

Accordingly, one aspect of the present disclosure provides a method of suppressing a T cell response in a subject in need thereof. Such method comprises reprogramming a target cell in the subject according to any of the methods disclosed above and herein. By way of non-limiting example, suppression of a T cell response may be suppression of activation of helper T cells in the subject.

Another aspect of the present disclosure provides a method of reducing or inhibiting an immune response in a subject in need thereof. Such method comprises reprogramming a target cell in the subject according to any of the methods disclosed above and herein.

Another aspect of the present disclosure provides a method of reducing or inhibiting an inflammatory response in a subject in need thereof. Such method comprises reprogramming a target cell in the subject according to any of the methods disclosed above and herein. By way of non-limiting example, the subject may be suffering from a lung inflammation, neuroinflammation, rheumatoid arthritis, and/or a primary immunodeficiency.

Another aspect of the present disclosure provides a method of reducing an immune response to a gene therapy regime in a subject in need thereof. Such method comprises reprogramming a target cell in the subject according to any of the methods disclosed above and herein.

Still another aspect of the present disclosure provides a pharmaceutical composition comprising processing bodies (p-bodies). By way of non-limiting example, the p-bodies are present within or isolated from mesenchymal stromal cells (MSCs). The MSCs may be derived from human tissues and/or cells, including, but not limited to, umbilical cord tissue (hCT-MSCs), bone marrow, adipose tissue, and induced pluripotent stem cells (iPSCs).

In some embodiments, the pharmaceutical composition may further comprise a pharmaceutically acceptable carrier. Any suitable pharmaceutically acceptable carrier may be used in the compositions of the disclosure. By way of non-limiting example, the pharmaceutically acceptable carrier may be Ringer's solution, Tyrode's solution, or a saline solution.

Still another aspect of the present disclosure provides a method of reprogramming a target cell by contacting the target cell with the pharmaceutical composition disclosed above and herein. In some embodiments, the target cell is a myeloid cell or a population of myeloid cells. By way of non-limiting example, the myeloid cell is a monocyte, a macrophage, or a dendritic cell.

Still another aspect of the present disclosure provides a method of suppressing a T cell response in a subject in need thereof. Such method comprises administering to the subject a therapeutically effective amount of the pharmaceutical composition disclosed above and herein. By way of non-limiting example, suppression of a T cell response may be suppression of activation of helper T cells in the subject.

Still another aspect of the present disclosure provides a method of reducing or inhibiting an immune response in a subject in need thereof. Such method comprises administering to the subject a therapeutically effective amount of the pharmaceutical composition disclosed above and herein.

Still another aspect of the present disclosure provides a method of reducing or inhibiting an inflammatory response in a subject in need thereof. Such method comprises administering to the subject a therapeutically effective amount of the pharmaceutical composition disclosed above and herein. By way of non-limiting example, the subject may suffer from a lung inflammation, neuroinflammation, rheumatoid arthritis, and/or a primary immunodeficiency.

Still another aspect of the present disclosure provides a method of reducing an immune response to a gene therapy regime in a subject in need thereof. Such method comprises reprogramming a target cell in the subject according to any of the methods disclosed above and herein.

Further aspect of the present disclosure provides a kit comprising the pharmaceutical composition disclosed above and herein and an instruction manual. By way of non-limiting example, such kit may be used for suppressing a T cell response, reducing or inhibiting an immune response, reducing or inhibiting an inflammatory response, and reducing an immune response to a gene therapy regime. Such kit would deliver p-bodies to myeloid cells, which engulf the p-bodies and undergo transcriptional reprogramming. The RNA transcripts expressed by the myeloid cells are altered as a result.

Certain aspects of the disclosure are now explained further via the following non-limiting examples.

### EXAMPLES

### Cell Culture

hCT-MSCs were obtained from the Robertson GMP lab at Duke. Umbilical cords were drained, cleaned with chlorhexidine gluconate, and separated from the placenta. Under GMP conditions, the cord was cut into small pieces and digested on a gentleMACS™ Octo Dissociator in buffer containing hyaluronidase, DNase, collagenase, and papain. Cell suspensions were plated on CellBIND® 75T flasks (Corning, NY, USA) in Prime-XV MSC Expansion XSFM (FUJIFILM Irvine Scientific, Inc., Santa Ana, CA, USA) and incubated in 37° C., 5% CO₂. Cells were detached using TrypLe™ Select Enzyme 10× (Thermofisher, Waltham, MA, USA) and collected for further experiment.

### hCT-MSC Differentiation

For adipocyte differentiation, Human Mesenchymal Stem Cell Functional Identification Kit (R&D) was used according to the manufacturer's instructions. hCT-MSC was plated into fibronectin treated plates in alpha MEM basal medium and grown for 1-3 days until the cells are 100% confluent, the cells are induced to undergo adipogenesis by the addition of Adipogenic supplement to the alpha MEM basal medium and media was replaced every 3-4 days. To confirm hCT-MSCs were differentiated to adipocyte, cells were stained with Fatty Acid Binding Protein 4 (FABP4) and then the secondary fluorescent antibody and Hoechst stain to assess differentiation into adipocyte.

For osteocyte differentiation, StemPro™ Osteogenesis Differentiation Kit (ThermoFisher) was used according to the manufacturer's instructions. hCT-MSCs were plated into fibronectin treated plates XSFM and grown for 1-2 days. When the cells are 50-70% confluent, the cells are induced to undergo osteogenesis by the addition of osteogenic differentiation medium. After the differentiation, cells were stained using anti-osteocalcin antibody.

For chondrocyte differentiation, StemPro™ Chondrogenesis Differentiation Kit (ThermoFisher) was used according to the manufacturer's instructions. Briefly, the cells are plated in micromass cultures into fibronectin treated plates XSFM medium and settled in well for 10 minutes at 37° C. incubator then add 0.5 ml Chondrogenesis differentiation medium. The media was changed every 2-3 days.

### T Cell Proliferation Assay

For the proliferation suppression assay, mouse splenocytes were co-cultured with hCT-MSCs (1:10=hCT-MSC:splenocytes) in RPMI 1640 containing 10% FBS, 1% penicillin/streptomycin and 25 mM HEPES with Dynabeads™ Mouse T-Activator CD3/CD28 (ThermoFisher). For irradiating hCT-MSCs, cells were dosed with 25 Gy, using a cesium irradiator. 33,000 cells were suspended in Prime-XV MSC Expansion XSFM and plated on fibronectin coated 96-well plates then placed in an incubator (37° C. 5% CO₂). After 48 hours, 1×10⁵ peripheral blood mononuclear cells (PBMCs) were suspended in RPMI 1640 containing 10% FBS, 1% penicillin/streptomycin and 25 mM HEPES, then plated on hCT-MSCs, and stimulated using Dynabeads human T activator CD3/28 (ThermoFisher). After 3 days, 3H thymidine (PerkinElmer, Waltham, MA, USA) was added in each well for a final concentration of 10 μCi/ml and after another 6-10 hour incubation, samples were collected using Perkin Elmer Filtermate harvester (PerkinElmer). 3H thymidine incorporation in human PBMCs was measured by a Microbeta Trilux 1450 LSC (PerkinElmer). For isolating T cells, a Pan T Cell Isolation Kit II (Miltenyi, Bergisch Gladbach, Germany) on a magnetic column was used to select T cells from mouse spleen and lymph nodes. The selected cells were suspended in serum-free RPMI 1640 with 5 μM of Carboxyfluorescein succinimidyl ester (CFSE; Thermofisher) for 10 minutes followed by addition of 2× volume of serum-containing media to stop the reaction. The T cells were washed and resuspended in RPMI 1640 containing 10% FBS, 1% penicillin/streptomycin and 25 mM HEPES co-cultured with hCT-MSC (1:10=hCT-MSC: T cells) with Dynabeads™ Mouse T-Activator CD3/CD28 (Thermofisher). For preconditioning, T cell-depleted splenoytes were prepared using a CD3e microbead kit (Miltenyi) and magnetic separation column, and the cells were preconditioned in a 0.4 μm pore transwell (MilliporeSigma, Burlington, MA, USA) or directly loaded on plated MSCs (1:10=hCT-MSC:splenocytes) for 72 hours. Control cells were similarly added to Prime-XV MSC Expansion XSFM for 72 hours. Preconditioned cells were isolated and co-cultured with CFSE labelled mouse T cell (1:3=preconditioned cell:T cells) with Dynabeads™ Mouse T-Activator CD3/CD28 stimulation. After 72 hours, cells were collected for evaluating proliferation. T cells were treated with Mouse BD Fc Block™ (BD bioscience) washed and stained with anti-CD4 APC-H7 (BD Bioscience, Franklin Lakes, New Jersey, USA) antibody and analyzed using a FACSLyric™ (BD Bioscience) and data were analyzed using BD FACSuite™ software (BD Bioscience). Qtracker™ labeled thymocyte and MSCs were cultured in EdU (ThermoFisher) containing media overnight and on the next day, apoptosis was induced by treating thymocytes with 50 μM dexamethasone and hCT-MSCs with 10 μg/ml cycloheximide with 20 ng/mL TNF-α. Cells were washed and cocultured with T cell-depleted splenocyte for 1 day and stained with anti-CD11b and Ly6G. Cells were fixed and incorporated Edu was labelled with Alexa Flour 488™. Cells were analyzed using FACSLyric™ and data were analyzed using BD FACSuite™ software.

### Phagocytosis Assay

Freshly thawed MSCs were labelled with QTraker™ and EdU according to the manufacturer's instructions then 2×105 cells were plated on fibronectin coated 6-well plates. After 24 hours, the cells were co-cultured with T cell-depleted splenocytes for 2 days and splenocytes were collected and stained with anti-B220 PerCPCy5.5, CD11b APC, CD11c Alexa 700, F4/80 PE-Cy7, Ly6C PE, and Ly6G APC-Cy7 (Biolegend, San Diego, CA, USA). Cells were analyzed using FACSLyric™. For EdU detection, cells were first collected and stained with CD11b APC and after that, cells were fixed, permeabilized and EdU in cells was labelled with Alexa488 according to the manufacturer's instruction.

Cells were analyzed using FACSLyric and data were analyzed using BD FACSuite™ software. For confocal imaging, hCT-MSCs were labelled with QTracker™ and plated on fibronectin coated plates for 1 day in RPMI 1640 containing 10% FBS, 1% penicillin/streptomycin and 25 mM HEPES. Then CD11B+ cells were sorted from splenocytes obtained from a C57BL/6-Tg (UBC-GFP)30Scha/J mouse and added to the hCT-MSCs. Images were obtained using Zeiss 780 inverted microscopy.

### RNA Sequencing and Analysis

QTracker labeled hCT-MSCs were plated on fibronectin coated 6-well plates and after 1 day, splenocytes were added, centrifuged briefly (1000 rpm, 5 minutes), then cultured for 3 days. Splenocytes were then collected and stained with anti CD11b, Ly6G antibodies. Qtracker positive or negative CD11b+Ly6C-cells were sorted using a MoFlo Astrios Cell Sorter (Beckman Coulter, Indianapolis, IN). RNA from sorted cells were purified by a RNeasy Mini kit (Qiagen, Hilden, Germany) according to manufacturer's instructions. cDNA libraries were generated using KAPA Stranded RNA-Seq Kits (Roche Sequencing Solutions) and sequenced on an Illumina HiSeq 4000. The raw sequencing reads (FASTQ files) were first chastity filtered, which removes any clusters that have a higher than expected intensity of the called base compared to other bases. They were then trimmed with Trimmomatic to remove low-quality bases (minimum read length after trimming). After preprocessing, the quality of the reads was evaluated using FastQC [10], and after passing quality control, the expression of the transcripts was quantified against the mm10 mouse genome (specifically, the Gencode M13 release) using Salmon. After quantification, the transcript abundances were then imported into R and summarized with tximport (giving gene level expression estimates), and then DESeq2 was used to normalize the raw counts, perform exploratory analysis (e.g., principal component analysis), and to perform differential expression analysis. The p-values from the differential expression analysis were corrected for multiple hypothesis testing (giving an adjusted p-value) with the Benjamini-Hochberg false-discovery rate procedure. Using the differentially expressed genes, the functional terms enriched in the samples was then determined with Fisher's exact test as implemented in the clusterProfiler Bioconductor package. The gene sets used for this analysis were from the Gene Ontology (GO) and Kyoto Encyclopedia of Genes and Genomes (KEGG).

### Real-Time PCR

After coculture with QTracker labeled hCT-MSCs splenocytes were then collected and stained with anti CD11b, Ly6G antibodies. Qtracker positive or negative CD11b+Ly6C− cells were sorted using a MoFlo Astrios Cell Sorter. RNA from sorted cells were purified by a RNeasy Mini kit and then, cDNAs were synthesized using SuperScript™ VILO™ cDNA Synthesis Kit (Thermofisher). Real-time PCR was performed on C1000 Touch Thermal Cycler (Bio-Rad) with TaqMan™ Fast Advanced Master Mix (Applied Biosystems, Foster City, CA). The primers (Thermofisher) used in this study were as follows: Ciita, Mm00482914_m1; H2-DMb2, Mm00783707_s1; Ox-40L, Mm00437214_m1; Rfx5, Mm01263513_g1.

### Phagocytosis Inhibitors

For blocker test, hCT-MSCs were labelled using Qtracker and plated on fibronectin-coated 24 well plate. One day after, T cell-depleted splenocytes were pre-incubated for 1 hour in 37° C., 5% CO₂ in each blocker containing culture media in following concentration: Mer RTK Inhibitor UNC569 (MilliporeSigma): 500 nM; Anti-TREM-2 antibody (MilliporeSigma): 2 μg/mL; Anti-Integrin α9 antibody (MilliporeSigma): 1 μg/mL; GST-receptor-associated protein: 250 nM. After that, cells were collected and resuspended in blocker containing media with the same concentration and then plated on hCT-MSC. Cell plates were briefly centrifuged and incubated for 2 days in 37° C., 5% CO₂. Cells were collected and stained with anti-CD11b antibody and then analyzed by FACSLyric™.

### Mice

Specific pathogen-free C57BL/6 mice were purchased from The Jackson Laboratory. All experiments were performed in accordance with Duke University Institutional Animal Care and Use Committee's policies.

### Generating DDX6 KO hCT-MSCs

DDX6 knockout hCT-MSCs were manufactured in the Duke Functional Genomics Shared Resource Core. To generate DDX6 knock out cell using CRISPR/Cas9 technology, sgRNA targeting DDX6 exon 3 was cloned into Cas9 expressing AAV vector. The following oligonucleotides from sense strand were used for sgRNAs targeting DDX6 exon 3: TCTCTAGACCTGGTGATGAC (SEQ ID NO:01). For control hCT-MSCs, a non-targeting gRNA sequence was used: ATTACTCTGATCTCACTCATTT (SEQ ID NO:02). For viral transduction, hCT-MSC were incubated with AAVS1 containing culture supernatant and after 3 days, cells were treated with puromycin over 2 days for virally transduced cell selection. Media was changed and cells were cultured for further experiments. All work using recombinant DNA was approved and followed NIH guidelines.

### DCP1A Expression in hCT-MSC

For transfecting hCT-MSCs, cells were plated on fibronectin coated 24-well plates, and incubated in normal cell incubation condition (37° C., 5% CO₂) for two days until the cells reached ˜80% confluency. For transient expression of DCP1A or DDX6, 1 μg of DCP1A-GFP plasmid was mixed with Lipofectamine 3000® (Thermofisher) according to manufacturer instruction or DDX6-RFP plasmid was mixed with TransIT-2020 transfection reagent (Mirus Bio LLC) at the ratio of reagent:DNA at 3:1 and DNA-reagent mixture was added on hCT-MSC. After 48 hours, the media was changed with Prime-XV MSC Expansion XSFM. To determine if p-bodies were transferred to monocytes or macrophages, transfected hCT-MSCs were incubated with CD11b+ MAC sorted monocytes and macrophages isolated from UBC-GFP mouse spleens for 4 hours. GFP+ monocytes and macrophages were then transferre to a new plate for imaging. DCP1A-GFP or DDX6-RFP punta in hCT-MSC or monocytes and macrophage were detected using Leica SP8 upright confocal microscopy (Leica Microsystems).

### Western Blot

Control or DDX6 KO hCT-MSC were lysed in protease inhibitor (MilliporeSigma) containing RIPA lysis buffer (MilliporeSigma) and separated using SDS polyacrylamide gel. The protein was transferred to PVDF membrane and blocked with 5% skim milk in PBS containing 0.05% Tween 20 and blotted using anti-DDX6 and B-actin antibodies (Abcam, Cambridge, UK). Subsequently, the blots were developed using the ECL Detection Kit (Bio-Rad Laboratories, Hercules, CA, USA) and protein bands were visualized using C-digit blot scanner (LI-COR Biosciences, Lincoln, NE, USA).

### Immunohistochemistry

hCT-MSCs were plated on fibronectin coated cover slides and incubated for 2 days. After, cells were fixed using 4% paraformaldehyde and incubated with blocking buffer (5% normal goat serum, 2% BSA, and 0.1% Triton X-100) in PBS) and stained using anti-DDX6 (abcam) and anti-Dcp1a antibody (Santa Cruz Biotechnology, Inc., Dallas, TX, USA). The cells were incubated with Alexa 488-conjugated secondary antibodies and mounted with VectaShield medium (Vector Labs, Burlingame, CA, USA). The images were acquired using Leica SP8 upright confocal microscopy (Leica Microsystems, Inc., Weltzlar, Germany). Z-stacks were analyzed by Image J for orthogonal views or Imaris Bitplane software 9.1.2 for 3D reconstruction.

### LPS-Induced Lung Inflammation

8 weeks old mice received vehicle, control hCT-MSC or DDX6 KO hCT-MSC (2×106 cells/150 μl) through iv injection 2 hours prior to LPS (10 μg/50 μl) intranasal administration. 24 h after LPS administration, mice were sacrificed, and lungs were collected for further analysis. Lungs were dissected into small pieces and enzymatically digested with liberase (0.2 mg/ml; MilliporeSigma) and DNase I (0.1 mg/ml) at 37° C. for 30 min in serum free RPMI and filtered by 70 μ cell strainer. The cells were treated with Mouse BD Fc Block™ and then stained by anti-CD3, CD11b, CD11c, Siglec F, I-A/I-E (biolegend) and 7AAD. Cells were analyzed using FACSLyric™ and data were analyzed using BD FACSuite™ software

### Statistics

Statistical analysis using a 1-way ANOVA followed by a Bonferroni post-hoc analysis was performed using Prism software (GraphPad Software Inc., La Jolla, CA, USA) unless otherwise described herein.

### Data Sharing

The data that support the findings of this study are available from the corresponding author upon reasonable request. Sequencing data has been uploaded to the GEO repository under Accession No. GSE149848.

### Example 1: hCT-MSCs Suppress T Cell Activation Via Programming Myeloid Cell

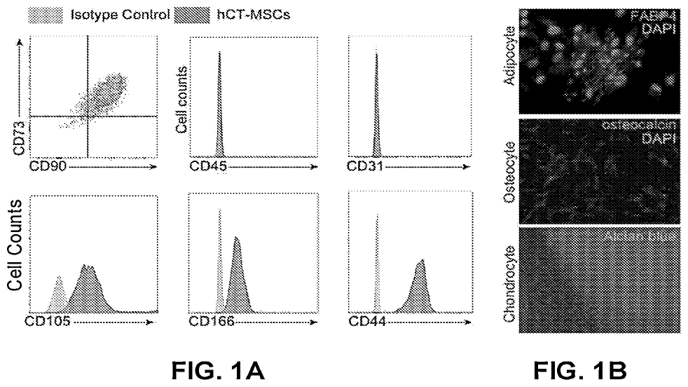

hCT-MSCs isolated from umbilical cord tissue was cultured, and their expression of canonical MSC markers was confirmed by flow cytometry (FIG. 1A). As shown in FIG. 1B, the hCT-MSCs had the potential to differentiate to adipocytes, osteocytes and chondrocytes.

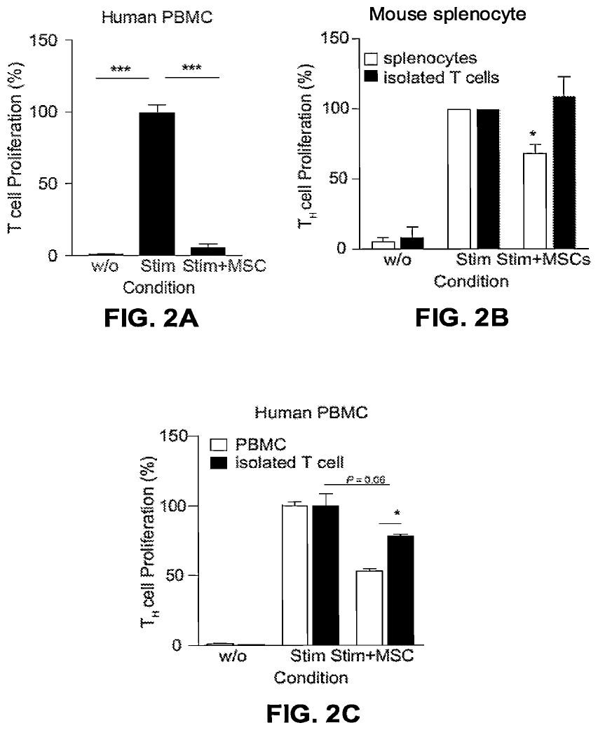

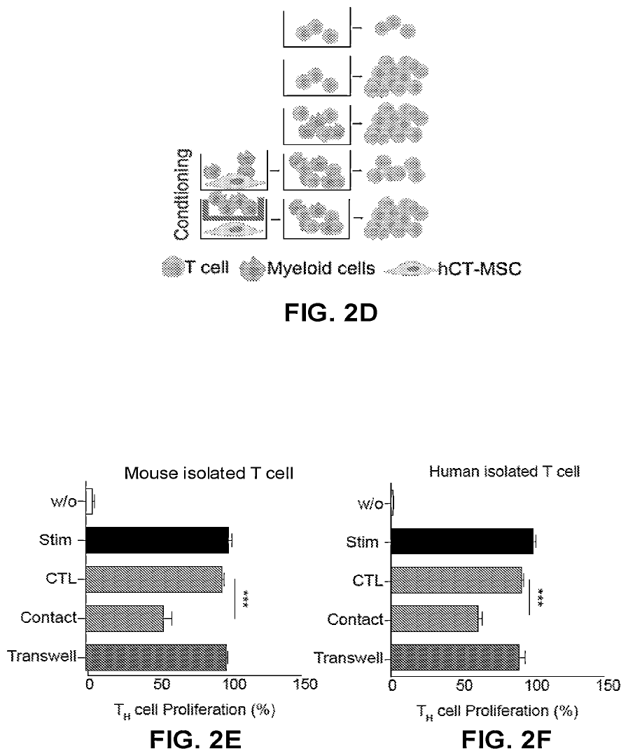

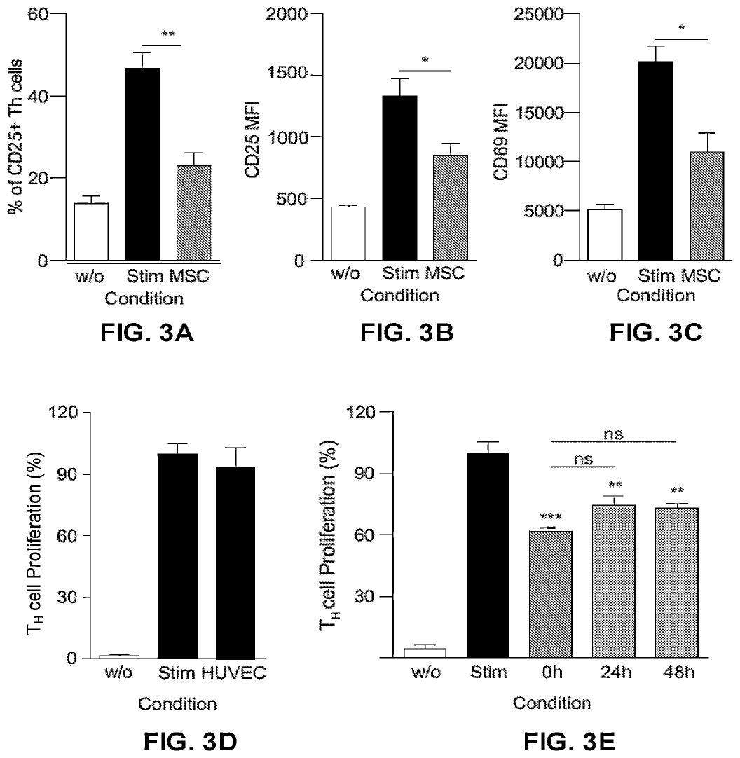

As shown in FIGS. 1A-1B, hCT-MSCs were not contaminated by endothelial cells (CD31+) or hematopoietic cells (CD45+). To test if they could inhibit a T cell response, hCT-MSCs was co-cultured with human peripheral blood mononuclear cells (PBMCs) stimulated with anti-CD3/28 antibodies. The results show that hCT-MSCs inhibited T cell proliferation (FIG. 2A) and suppressed the expression of activation markers CD25 and CD69 on helper T cells (TH) (FIGS. 3A-3C).

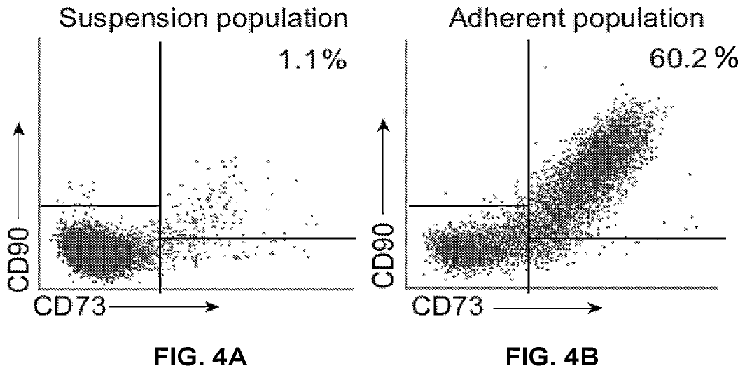

hCT-MSC also suppressed TH cell activation in mouse splenocytes. Interestingly, although hCT-MSCs inhibited T cells within a bulk splenocyte pool, hCT-MSCs failed to inhibit the proliferation of isolated TH cells suggesting that a non-T cell is needed for the immune suppression function of hCT-MSCs (FIG. 2B). Similar results were found in human blood where hCT-MSCs suppressed TH cells in PBMCs to a much great extent than isolated TH cells (FIG. 2C). Immunosuppression was specific to hCT-MSCs since human umbilical vein endothelial cells (HUVECs) did not affect the proliferation of TH cells (FIG. 3D). Since in vivo data demonstrate that MSCs can have long-lasting beneficial effects despite being quickly cleared, and MSC-primed monocytes can induce regulatory T cell populations, it was hypothesized that MSCs must interact with myeloid cells to successfully inhibit T cells. To test this, mouse splenocytes or human PBMC were preconditioned, depleted of T cells, with hCT-MSCs prior to co-culturing with activated T cells (FIG. 2D). Both mouse splenocytes and human PBMC, depleted of T cells and preconditioned with hCT-MSC inhibited TH cell proliferation in the absence of hCT-MSCs (FIGS. 2E-2F). It was confirmed that hCT-MSCs did not transfer over to the T cell proliferation assay (FIG. 4A) and the ability for preconditioned cells to suppress TH cells was retained for at least 48 hours (FIG. 3E).

In order to be preconditioned, myeloid populations must physically interact with the hCT-MSCs since the effect was abolished if the cells were separated by a transwell filter (FIGS. 2D-2E). These data suggest that direct cell-to-cell contact is critical for hCT-MSCs to reprogram myeloid populations.

### Example 2: Engulfment of hCT-MSCs is Critical to Reprogramming Monocytes and Macrophages

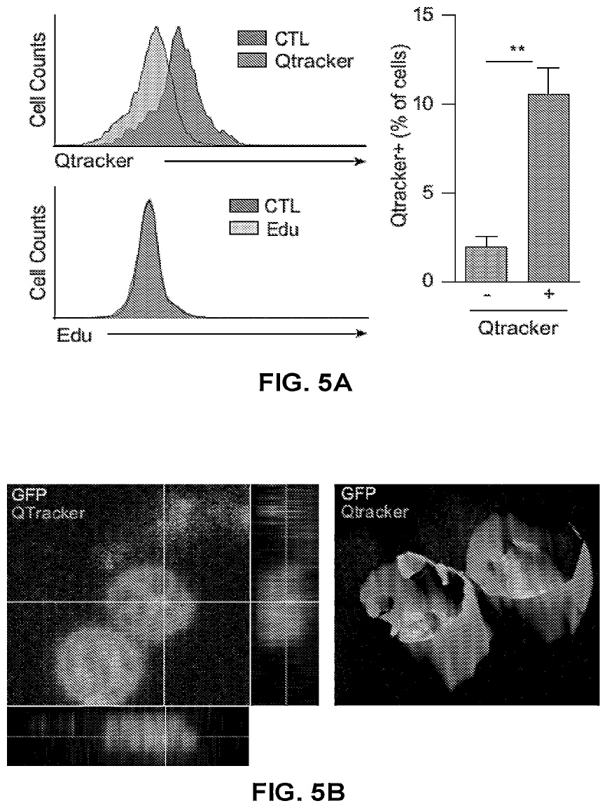

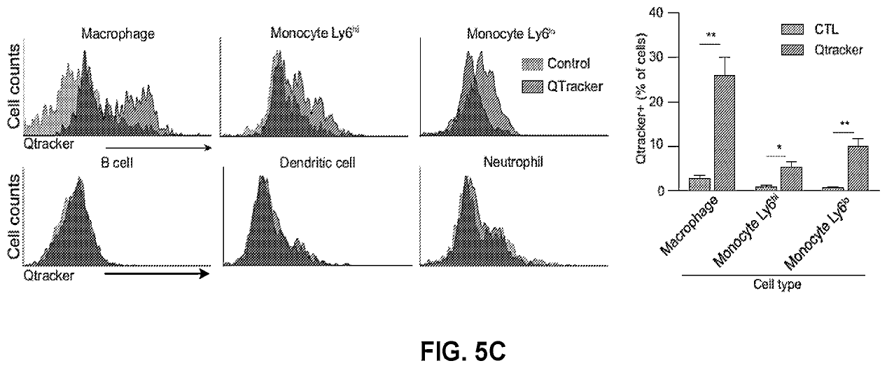

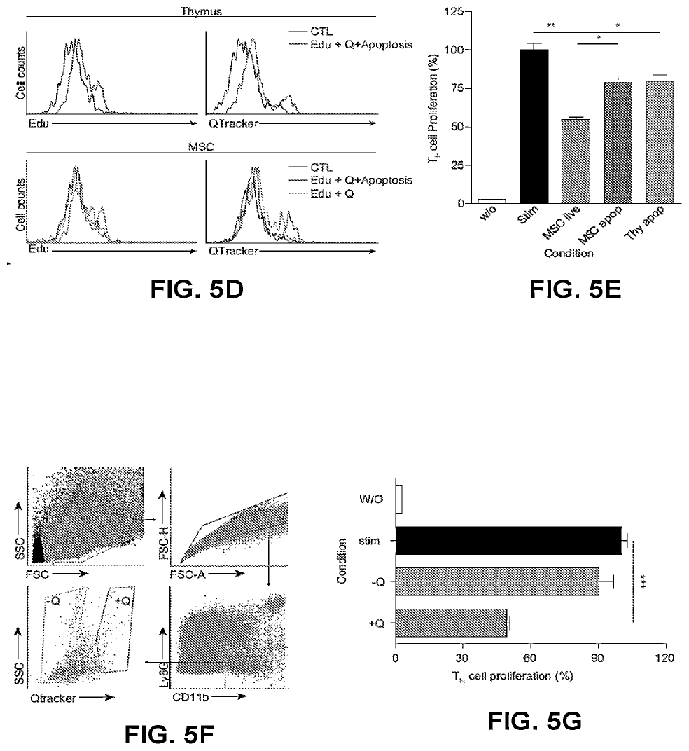

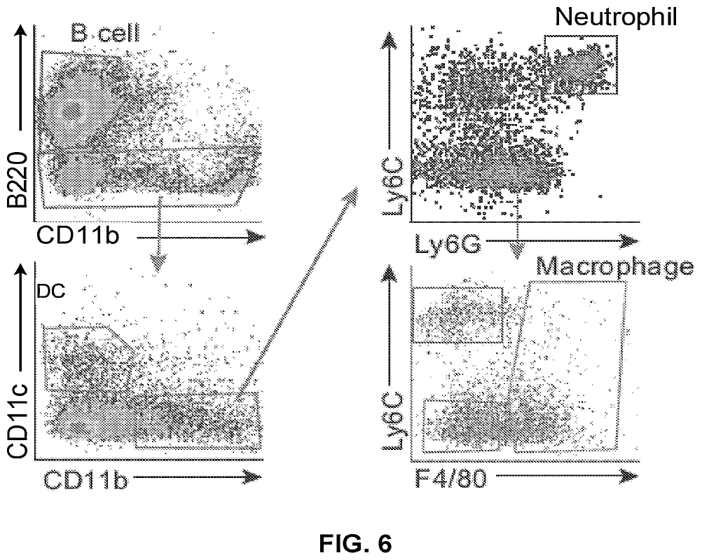

MSCs injected intravenously in mice are rapidly cleared within 24 hours. Because their remnants can be detected in CD11b+ myeloid cells, it was hypothesized that CD11b+ myeloid cells with direct cell-to-cell contact would engulf hCT-MSCs. To address this, the cytoplasm of hCT-MSCs was labelled with Qtracker and the nucleus with Edu, then the ability of splenic myeloid cells to engulf live hCT-MSCs was tested. Like others, CD11b+/Qtracker+ myeloid cells were detected 2 days after being incubated with labelled hCT-MSCs (FIGS. 5A-5B). However, EdU was not detected in any splenocytes (FIG. 5A). Since EdU was incorporated into DNA during replication while Qtracker was restricted to the cytosol, it was concluded that myeloid cells only engulfed cytosolic components of live hCT-MSCs but did not phagocytosis the entire cell. Furthermore, it was identified that the cells which engulfed hCT-MSCs were mostly macrophages and Ly6Clo resident monocytes, with a smaller percent of Ly6Chi inflammatory monocytes positive for Qtracker (FIGS. 5C; FIG. 6).

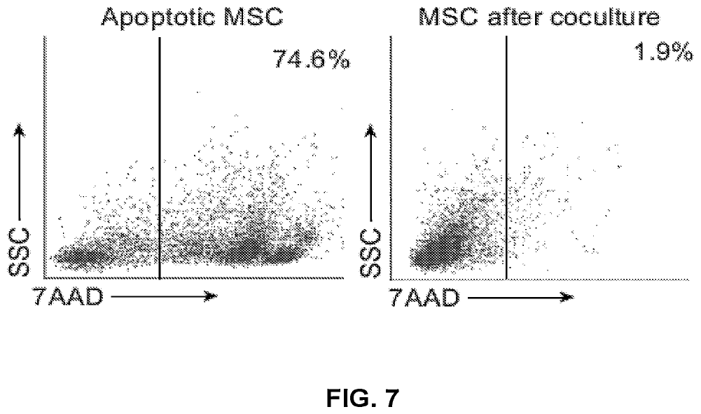

Phagocytosis of apoptotic cells (i.e., efferocytosis) can skew professional phagocytes to an anti-inflammatory phenotype. Therefore, it was tested if monocytes and macrophages incubated with apoptotic hCT-MSCs or apoptotic thymocytes would also inhibit the activation of TH cells. Unlike when incubated with live hCT-MSCs, monocytes and macrophages phagocytosed whole apoptotic hCT-MSCs and thymocytes, as expected, indicated by EdU positive signal in macrophages (FIGS. 5D; FIG. 7).

In agreement with other studies, efferocytosis of either type of apoptotic cells resulted in monocytes and macrophages suppressing the proliferation of T cells; however, neither suppressed to the same extent as macrophages that engulfed cytoplasmic components of live hCT-MSCs (FIG. 5E). To test if engulfing cytoplasmic components of hCT-MSCs is a critical process to reprogramming monocytes and macrophages. Qtracker positive or negative monocytes and macrophages were isolated by fluorescence-activated cell sorting (FACS) after they were cocultured with live labeled hCT-MSCs (FIG. 5F). The results show that Qtracker+ monocytes and macrophages inhibited TH cell proliferation while Qtracker− cells failed to inhibited T cell proliferation (FIG. 5G).

### Example 3: Transcriptional and Functional Alterations in Monocytes and Macrophages After Engulfing Components of hCT-MSCs

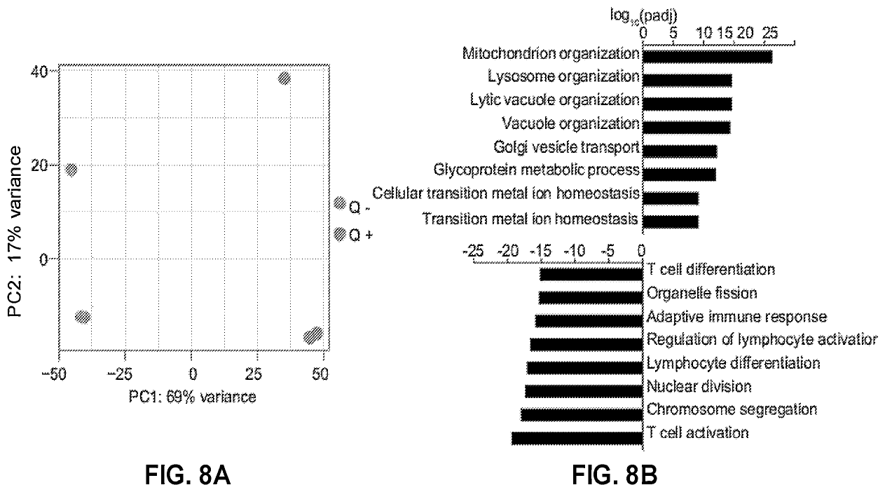

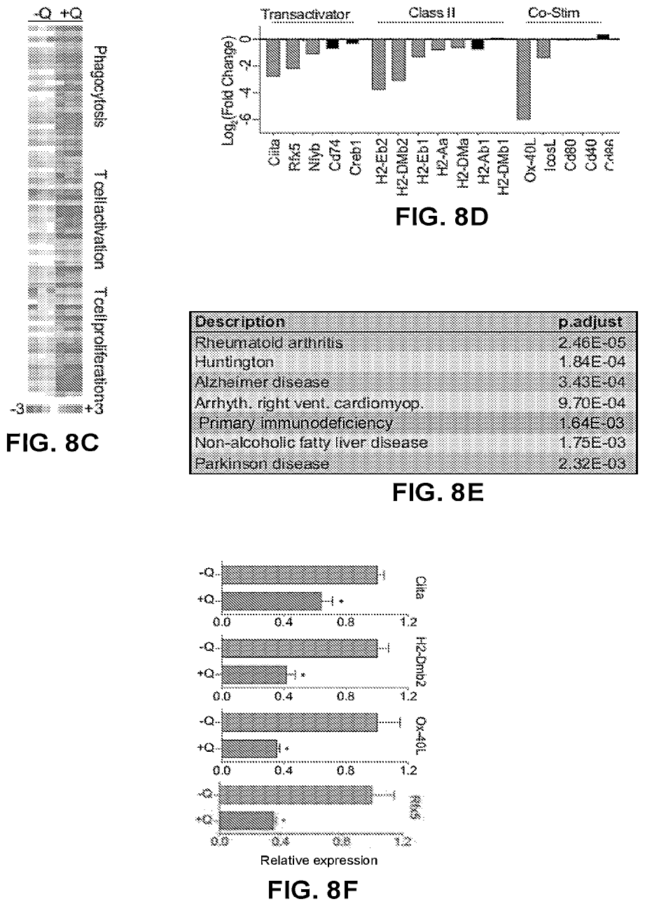

To understand functional changes monocytes and macrophages undergo after engulfing components of live hCT-MSCs, RNA of Qtracker positive or negative monocytes and macrophages were FACS sorted and sequenced 48 h after coculture with hCT-MSCs. Principal component analysis of statistically significant transcripts revealed that the two populations were transcriptionally distinct (FIG. 8A).

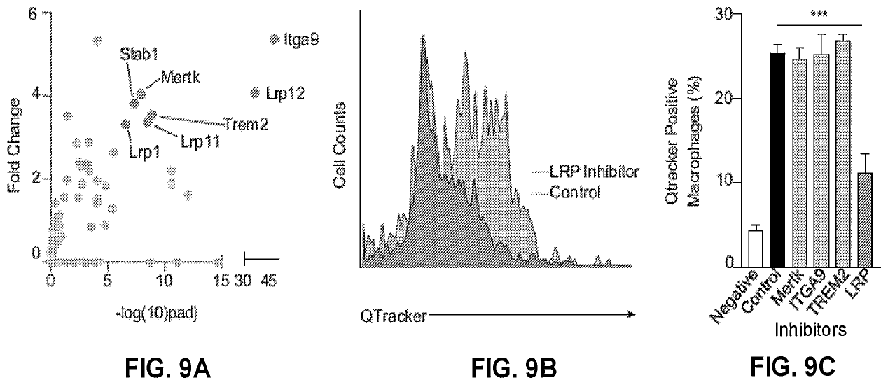

To understand functional differences of these populations, a gene set enrichment analysis was performed. It was determined that the top gene sets down-regulated in Qtracker positive monocytes and macrophages were associated with the activation of T cells, in particular genes associated with antigen presentation, while up-regulated gene sets were associated with cellular organization (FIGS. 8B-8F). The mostly highly enriched disease sets included rheumatoid arthritis and primary immunodeficiency (FIG. 8E). With these data, it was concluded that monocytes and macrophages that engulf cytoplasmic components of hCT-MSC are transcription poised to inhibit the activation of TH cells. Phagocytosis and other cell engulfment pathways are mediated by ligand-receptor interactions. To determine a potential receptor on monocytes and macrophages needed to engulf cytoplasmic components of MSCs, molecules involved in cell-to-cell interactions were curated and the gene expression of these molecules upregulated in Qtracker+ monocytes and macrophages was plotted (FIG. 9A).

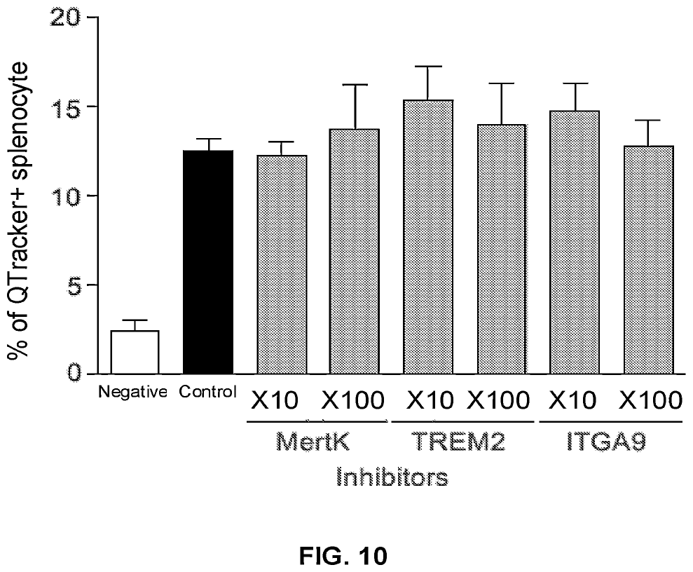

Pharmacological inhibitors for 6 of the 7 top hits were identified: Low density lipoprotein receptor-related proteins (LRP1, 11, and 12). MER Proto-Oncogene, Tyrosine Kinase (MERTK), integrin subunit alpha 9 (ITGA9), and Triggering receptor expressed on myeloid cells 2 (TREM2). Only receptor associated protein (RAP), a pan inhibitor or LRPs was able to inhibit monocytes and macrophages from engulfing cytoplasmic components of hCT-MSC (FIGS. 9B-9C; FIG. 10).

These data suggest that the interaction between monocytes and macrophages and hCT-MSCs is mediated through LRPs and results in monocytes and macrophages engulfing cytoplasmic components of hCT-MSCs.

### Example 4: Process Body-Dependent Reprogramming of Macrophages

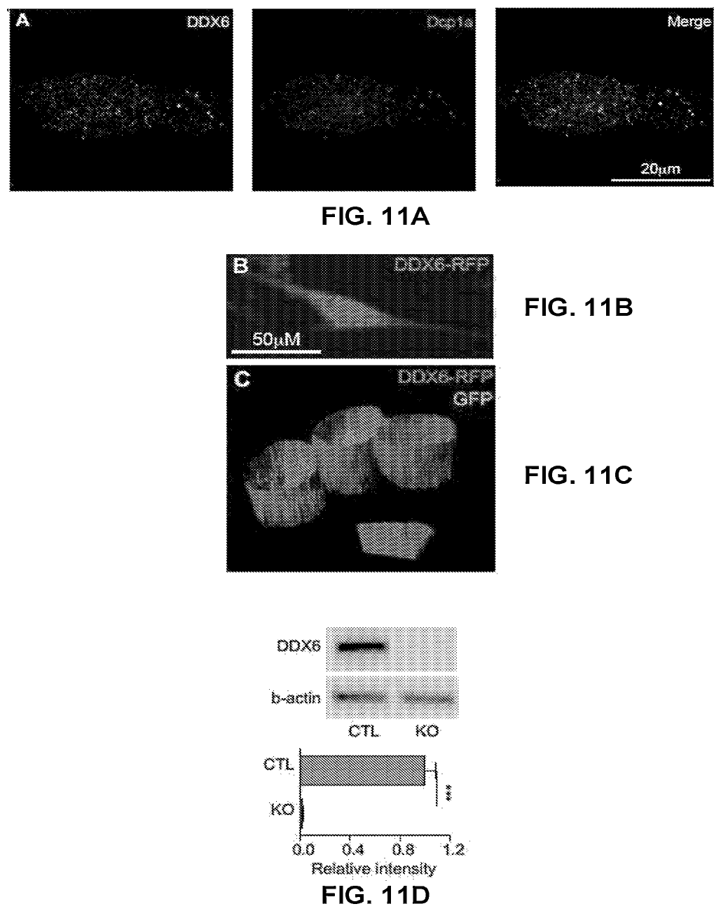

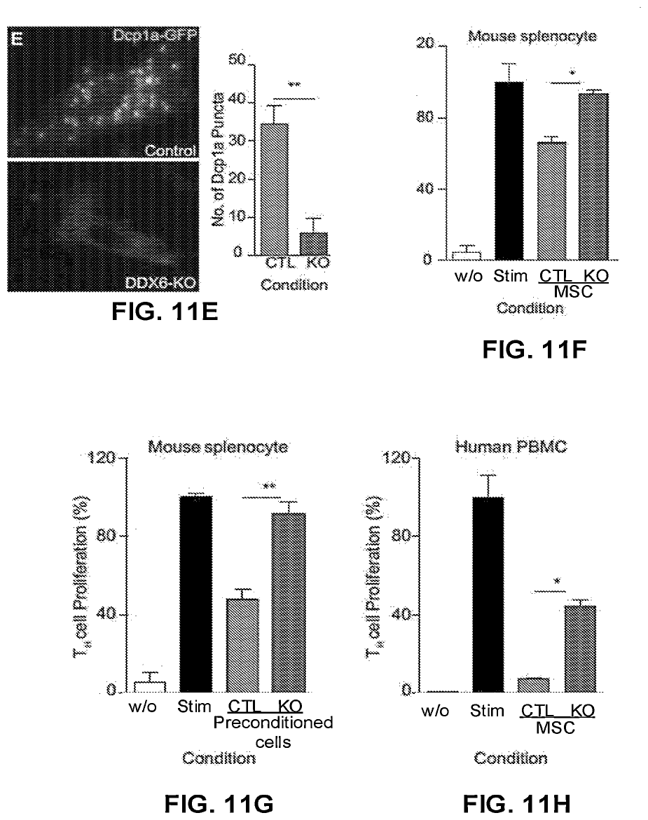

Given that monocytes and macrophages must engulf cytoplasmic components to suppress TH cells, potential mechanisms how cells could package and transfer large amounts of information via cytoplasm were explored. Processing bodies (p-bodies) are cytosolic membrane-less organelles that store RNA, miRNA, and proteins. Using antibodies detecting DCP1A (decapping mRNA 1A) or DDX6 (DEAD-Box Helicase), major components of p-bodies, it was found that p-bodies are abundant in hCT-MSCs (FIG. 11A).

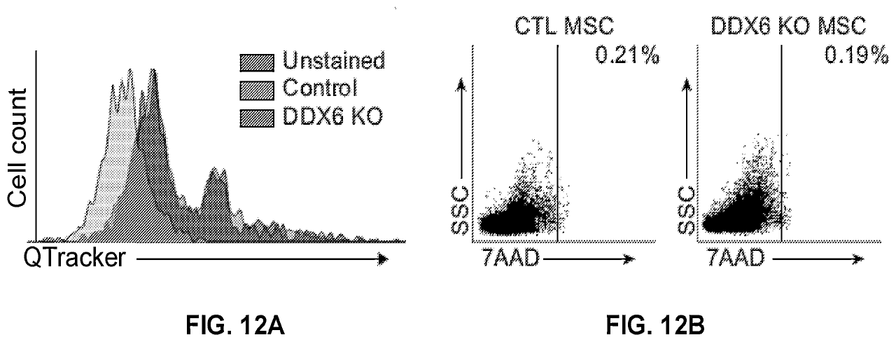

It was hypothesized that p-bodies in hCT-MSCs function to store packaged information to be transferred to functionally reprogram monocytes and macrophages. To address this, it was first tested if p-bodies from hCT-MSC were transferred to monocytes and macrophages. After coculturing monocytes and macrophages with hCT-MSC that transiently overexpressed DDX6-RFP, DDX6-RFP signal was detected in the monocytes and macrophages (FIGS. 11B-11C). These data suggest that p-bodies in hCT-MSC can be transferred to monocytes and macrophages. Next. DDX6 knockout hCT-MSC cells were generated with CRISPR/Cas9 (FIG. 11D). DDX6 is an RNA binding protein critical to stabilize p-bodies. As expected, DDX6 knockout hCT-MSCs failed to produce p-bodies (FIG. 11E). Although monocytes and macrophages could still engulf components of DDX6-knockout hCT-MCSs (FIG. 12A), they failed to program monocytes and macrophages and suppress the proliferation of human and mouse TH cells (FIGS. 11F-11H). To note, no changes in viability of DDX6 knockout hCT-MSCs were detected (FIG. 12B).

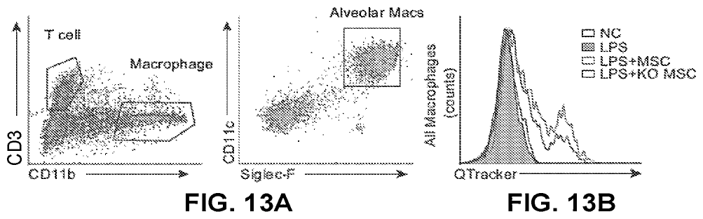

It was concluded that p-bodies within hCT-MSCs were a critical component to reprogramming monocytes and macrophages to inhibit the activation of TH cells. P-bodies in hCT-MSCs are critical to suppress inflammation in the lung. To test the function of hCT-MSC p-bodies in vivo, lung inflammation was induced with intranasal lipopolysaccharide (LPS). Two hours before administering LPS, control hCT-MSCs or DDX6 KO hCT-MSCs was injected (IV). To track hCT-MSCs, the cells were labelled with QTracker. Twenty-four hours after LPS, Qtracker signal was detected within lung monocytes and macrophages, suggesting they engulfed hCT-MSCs (FIG. 13B).

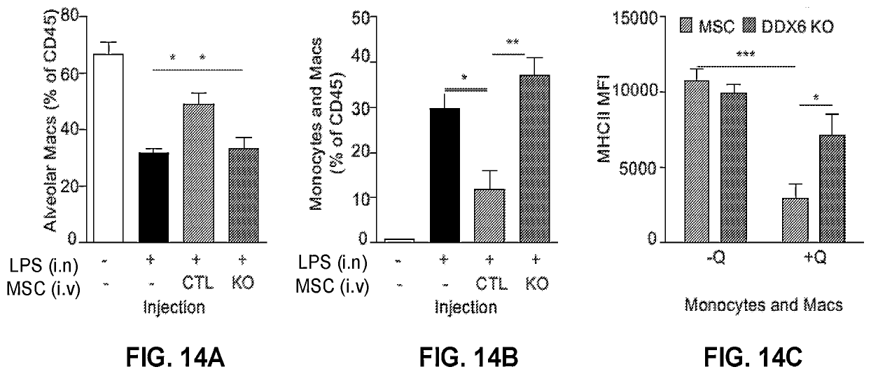

The results further show that DDX6 KO hCT-MSCs were engulfed to the same extend as control hCT-MSCs. Functionally, hCT-MSCs blocked an LPS-induced loss of CD11c+ alveolar macrophages (FIG. 14A) and an increase in CD11b+ inflammatory monocytes and macrophages populations in lung tissue (FIG. 14B). Importantly, CD11b+ monocytes and macrophages that engulfed components of hCT-MSC had decreased surface expression of MHC-II (FIG. 14C). Compared to control hCT-MSCs, DDX6 KO hCT-MSCs failed to block a loss of alveolar macrophages or an influx in inflammatory CD11b+ cells in the lung. (FIGS. 14A-14B). Furthermore, MHC-II expression on CD11b+ monocytes and macrophages which engulfed DDX6 KO hCT-MSCs was not altered (FIG. 14C). These data demonstrate that p-bodies in hCT-MSC are critical component for suppressing inflammation.

Discussion: MSCs represent approximately 25% of all cell-based clinical trials with over 1,000 trials registered on US government website for clinical trials. MSCs are well documented to influence multiple immune cell populations yet how they confer benefit in vivo is unclear. Despite demonstrating long-lasting effects in vivo, MSCs do not engraft and are rapidly cleared. This enigma defines a critical barrier advancing MSCs as a reliable therapeutic option for inflammatory disease. The present disclosure demonstrates that hCT-MSCs indirectly suppress the activation of TH cells through interacting with monocytes and macrophages. This corroborates other findings demonstrating that monocytes and macrophages are necessary for the beneficial effects of MSCs in vitro. Likewise, the interactions in vivo between MSCs and monocytes and macrophages are critical to suppress a proinflammatory adaptive immune response. In a mouse model of graft-versus-host disease, MSCs prevented effector T cells from infiltrating the lungs and spleen. Labelled MSCs homed to the lungs were engulfed by monocytes and macrophages. Similarly, in mouse models of sepsis, labelled MSCs were engulfed by alveolar macrophages with increased survival. In both models, depleting macrophages with clodronate-filled liposomes blocked any beneficial effects of MSCs. It is presumed that once injected, MSCs undergo apoptosis then macrophages phagocytose the labelled MSC corpses (efferocytosis); however, an alternate mechanism was determined where monocytes and macrophages instead engulf cytoplasmic components of live MSCs (FIG. 5).

According to the studies (FIG. 2C), the ability of MSCs to reprogram monocytes and macrophages was dependent on direct contact. When monocytes and macrophages were separated from hCT-MSCs by a transwell membrane, they failed to suppress activated TH cells. The pore size (0.4 μM) in the transwells would not prevent the passage of exosomes (30-100 nm), suggesting reprograming depended on direct cellular contact. When hCT-MSC with Qtraker were co-labelled to label the cytoplasm and EdU to label the nucleus, it was found that monocytes and macrophages only took up Qtracker (FIG. 5A). Consequently, only cells that took up Qtracker from hCT-MSCs were reprogrammed to suppress TH cells (FIG. 5G). When apoptosis of Qtracker+/Edu co-labelled hCT-MSCs was induced, then monocytes and macrophages phagocytosed entire hCT-MSCs (i.e., cells took up Qtracker and EdU; FIG. 5D). Monocytes and macrophages, after efferocytosis of hCT-MSCs, attenuated T cell proliferation; however, the response was not as robust as after engulfing cytoplasmic components of live hCT-MSCs (FIG. 5E). Further, monocytes and macrophages attenuated TH cell proliferation to similar level after efferocytosis of apoptotic thymocytes. These data suggest two separate mechanisms driving anti-inflammatory responses in professional phagocytes: first, a non-specific anti-inflammatory response from efferocytosis, and second, a specific response that depends on contact-mediated transfer of cytoplasmic components of live hCT-MSCs. Although efferocytosis likely contributes to engulfing and clearing MSCs; trogocytosis (nibbling), paracytophagy, and tunneling nanotubes are also potential ways phagocytic myeloid cells can uptake components of live MSCs. After efferocytosis, trogocytosis, and paracytophagy cargo is encapsulated in intracellular vesicles with lipid bilayers. Either secondary signaling pathways in response to the process itself could be activated or intracellular cargo would need to be released from the vesicle to directly initiate signaling pathways. Tunneling nanotubes (TNTs) have been described in MSCs and macrophages and offer an alternative pathway. TNTs are cellular extensions that enable the transfer of cytosolic material from one cell to another cell through direct contact. A substantial amount of cytoplasmic cargo can be transferred through TNTs and they even demonstrated the ability to support transfer of RNA and large organelles such as mitochondria from MSCs to macrophages. Formation of TNTs and transfer of cargo from MSCs increased the phagocytotic activity of macrophages. After monocytes and macrophages engulf components of hCT-MSCs, they undergo transcriptional changes and continue to suppress T cells even when hCT-MSCs are no longer present (FIG. 5G).

Monocytes and macrophages that engulf components of hCT-MSCs down-regulated genes responsible for presenting antigens and activating T cells, including disease gene sets implicated in rheumatoid arthritis and primary immunodeficiencies (FIGS. 8B-8F; FIG. 14C). Using a combination of computational and pharmacological approaches, the transfer of Qtracker from hCT-MSCs to monocytes and macrophages was inhibited using a pan LRP inhibitor (FIG. 9). LRPs are endocytic receptors that not only control the uptake of lipoproteins but are also involved in clearing dead cells and modulating the inflammatory response. For example, deleting LRP expression on microglia (the brain's resident macrophages) enhanced pro-inflammatory cytokine secretion. The present disclosure suggests that LRP mediates cell-to-cell interaction between hCT-MSCs and monocytes and macrophages which results in reprogramming monocytes and macrophages to inhibit the activation of T cells. Considering its main function, LRP would function in the monocytes and macrophages membrane to initiate the uptake of cytoplasmic components of hCT-MSCs. Once engaged through LRPs, MSCs would need to transfer signals to monocytes and macrophages through cytoplasmic components. Since a large amount of transcriptional information can be packaged in cytoplasmic p-bodies, and p-bodies are necessary for the epithelial-to-mesenchymal transition, hCT-MSCs were first stained with DCP1A and DDX6 and it was found that p-bodies to be abundant (FIG. 11A). P-bodies are membrane-less, liquid-liquid phase cytoplasmic organelles that contain RNAs and RNA-binding proteins. P-bodies were initially thought to sequester mRNAs during stress; however, they have now been shown to play a role in translation under homeostasis, such as in synaptic plasticity. Over 33% of all coding mRNA can be packaged into p-bodies and the RNA is enriched for control regulatory functions. Moreover, in vitro experiments revealed that formation of p-bodies is critical to suppress inflammatory cytokine expression in endotoxin tolerant macrophages. When p-bodies in hCT-MSCs were genetically removed by deleting DDX6 via CRISPR/Cas9, DDX6-KO hCT-MSCs failed to reprogram monocytes and macrophages and suppress TH cells in vitro (FIGS. 11D-11G). In vivo data using an LPS-induced lung inflammation model demonstrated that DDX-KO hCT-MSC lost their ability to suppress inflammation and decrease the surface expression of MHC class II on monocytes and macrophages that engulfed MSCs (FIG. 14C). Deleting DDX6 did not prevent macrophages from engulfing cytoplasmic components of hCT-MSCs (FIG. 12A; FIG. 13B). The present disclosure suggests a novel mechanism regarding how MSCs can program monocytes and macrophages to inhibit TH cell activation and proliferation which can explain long-term effects from MSC even after they are cleared.

In conclusion, the present disclosure reports a novel mechanism of how hCT-MSCs reprogram monocytes and macrophages to suppress the activation of TH cells. hCT-MSCs directly contact monocytes and macrophages and transfer cytoplasmic components. The transfer of cytoplasmic material was dependent on LRP on the surface of monocytes and macrophages and processing bodies in hCT-MSCs. Monocytes and macrophages that engulfed hCT-MSC downregulated genes in antigen presentation and co-stimulatory pathways and could suppress the activation of T cells after hCT-MSCs were no longer present. These data could explain how MSCs have long-lasting beneficial effects in vivo despite being cleared hours after administration.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A pharmaceutical composition comprising processing bodies (p-bodies), wherein the p-bodies are present within or isolated from mesenchymal stromal cells (MSCs), and a pharmaceutically acceptable carrier.

2. (canceled)

3. The pharmaceutical composition of claim 1, wherein the MSCs are derived from umbilical cord tissue, bone marrow, adipose tissue, and/or induced pluripotent stem cells (iPSCs).

4. (canceled)

5. (canceled)

6. (canceled)

7. (canceled)

8. A method of suppressing a T cell response in a subject in need thereof, the method comprising administering to the subject a therapeutically effective amount of the pharmaceutical composition of claim 1.

9. The method of claim 8, wherein suppressing the T cell response comprises suppressing activation of helper T cells in the subject.

10. (canceled)

11. (canceled)

12. The method of claim 8, wherein the subject suffers a lung inflammation, neuroinflammation, rheumatoid arthritis, and/or a primary immunodeficiency.

13. (canceled)

14. (canceled)

15. A method of reprogramming a target cell, the method comprising contacting the target cell with one or more mesenchymal stromal stem cells (MSCs).

16. The method of claim 15, wherein the MSCs are derived from umbilical cord tissue, bone marrow, adipose tissue, and/or induced pluripotent stem cells (iPSCs).

17. The method of claim 16, wherein the target cell is a myeloid cell or a population of myeloid cells.

18. The method of claim 17, wherein the target myeloid cell or the population of target myeloid cells engulfs the MSCs via cell-to-cell interaction.

19. The method of claim 18, wherein the cell-to-cell interaction is mediated through lipoprotein receptor-related proteins (LRPs) on the surface of the target myeloid cell or the population of target myeloid cells.

20. The method of claim 18, further comprising manipulating the MSCs to target the delivery of the cytoplasmic components of the MSCs to the target myeloid cell or the population of target myeloid cells.

21. The method of claim 20, wherein the cytoplasmic components are processing bodies (p-bodies) within the MSCs.

22. The method of claim 17, wherein the target myeloid cell is a monocyte, a macrophage, or a dendritic cell, or wherein the population of target myeloid cells is a population of monocytes, macrophages, or dendritic cells.

23. (canceled)

24. (canceled)

25. (canceled)

26. (canceled)

27. The method of claim 29, wherein the subject suffers from a lung inflammation, neuroinflammation, rheumatoid arthritis, and/or a primary immunodeficiency.

28. (canceled)

29. The method of claim 17, wherein the target myeloid cell or the population of target myeloid cells is in a subject.

30. The method of claim 29, wherein the subject is receiving or has received a gene therapy regimen.


SEQUENCE LISTING (2 sequences)

SEQ ID NO: 1 - DNA, 20 bp, Artificial Sequence
  Synthetic
tctctagacctggtgatgac

SEQ ID NO: 2 - DNA, 22 bp, Artificial Sequence
  Synthetic
attactctgatctcactcattt